(19) United States
(12) Patent Application Publication
(10) Pub. No.: US 2020/0407392 A1
(43) Pub. Date: Dec. 31, 2020

(54) LNA-DICARBOXYLIC ACID DERIVATIVES AND PROCESS FOR THEIR PREPARATION

(71) Applicant: Hoffmann-La Roche Inc., Little Falls, NJ (US)

(72) Inventors: KURT PUENTENER, BASEL (CH); CHRISTOPH ROSENBOHM, HORSHOLM (DK); FILIPPO SLADOJEVICH, BASEL (CH)

(73) Assignee: Hoffmann-La Roche Inc., Little Falls, NJ (US)

(21) Appl. No.: 17/016,592

(22) Filed: Sep. 10, 2020

Related U.S. Application Data
(63) Continuation of application No. PCT/EP2019/056068, filed on Mar. 12, 2019.

(30) Foreign Application Priority Data
Mar. 14, 2018 (EP) 18161687.1

(51) Int. Cl. C07H 19/06; C07H 1/00; C07H 19/16

(52) U.S. Cl. CPC C07H 19/06; C07H 19/16; C07H 1/00

(57) ABSTRACT

The invention comprises LNA-dicarboxylic acid derivatives of the formula I

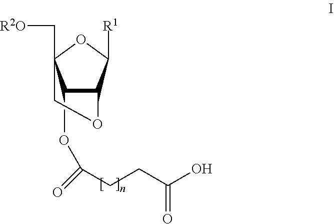

or salts thereof,
wherein
   R¹ is a nucleobase or a modified nucleobase
   R² is a hydroxy protecting group and
   n is an integer from 1 to 5.

The LNA-dicarboxylic acid derivatives of the formula I can be used in preloaded solid supports of the formula III

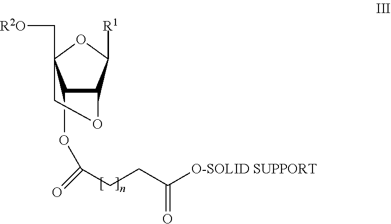

and as that serve as suitable start material for oligonucleotide synthesis.

### CROSS REFERENCE TO RELATED APPLICATIONS

This application is a continuation of International Application No. PCT/EP2019/056068 having an international filing date of Mar. 12, 2019, the entire contents of which are incorporated herein by reference, and which claims benefit under 35 U.S.C. § 119 to European Patent Application No. 18161687.1 filed on Mar. 14, 2018.

### SUMMARY

The invention relates to LNA-dicarboxylic acid derivatives of the formula I

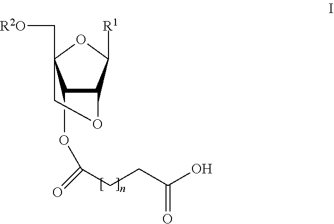

or salts thereof,
wherein
   R¹ is a nucleobase or a modified nucleobase
   R² is a hydroxy protecting group and
   n is an integer from 1 to 5
      to a process for their preparation, to its use for the preparation of a LNA-preloaded solid support containing the LNA-dicarboxylic acid derivatives of the formula I, to a LNA-preloaded solid support and its use in the oligonucleotide synthesis.

### DETAILED DESCRIPTION

Oligonucleotides are generally prepared on a solid support medium. In general a first synthon (e.g. a monomer, such as a nucleoside) is first attached to the solid support medium, and the oligonucleotide is then synthesized by sequentially coupling monomers to the solid support-bound synthon. This iterative elongation eventually results in a final oligonucleotide compound which it is cleaved from the support and, if necessary further worked up to produce the final oligonucleotide compound.

In a further development linker molecules attached to the solid support have been introduced for carrying out the oligonucleotide synthesis more efficiently, particularly on the automated process synthesizers nowadays used. For instance the Int. Patent Publication WO 2005/049621 or WO 2006/029023 discloses such linker compounds. Oligonucleotide elongation usually starts from an O-dimethoxytrityl (O-DMT) on the linker molecule.

However, in the synthesis of LNA oligonucleotides, it was found in that to a certain extent the first coupling on the linker molecule failed, resulting in final oligonucleotides with a substantial amount of (N−1) impurities, i.e. oligonucleotides with one lacking nucleoside at the 3′-end.

Object of the present invention therefore was to reduce the amount of 3′-end (N−1) impurities to a great extent, as such impurities can hardly be removed by state-of-the-art chromatographic purification methods and cause thereof a substantial loss in yield, e.g. by tight fraction pooling in the preparative chromatography step and/or an unsatisfying impurity profile in the isolated LNA oligonucleotide

It was found that with LNA-dicarboxylic acid derivatives of the formula I as described above, which can be attached to a solid resin (Int. Patent Publication WO 1992/006103) and act as pre-loaded resin the N−1 failures can be reduced to great extent.

The following definitions are set forth to illustrate and define the meaning and scope of the various terms used to describe the invention herein.

The term “C₁₋₆-alkyl” denotes a monovalent linear or branched saturated hydrocarbon group of 1 to 6 carbon atoms, and in a more particular embodiment 1 to 4 carbon atoms. Typical examples include methyl, ethyl, propyl, isopropyl, n-butyl, i-butyl, sec-butyl, or t-butyl, preferably methyl or ethyl.

The term “C₁₋₆-alkyoxy” denotes a C₁₋₆-alkyl group, more preferably a C₁₋₄-alkyl group, as defined above attached to an oxygen atom. Typical examples include methoxy, ethoxy, propoxy, isopropoxy, n-butoxy, i-butoxy, t-butoxy, preferably methoxy or ethoxy.

The term “C₁₋₆-alkanoyl” denotes a denotes a C₁₋₆-alkyl group, more preferably a C₁₋₄-alkyl group, as defined above attached to a carbonyl group Typical examples include acetyl, ethanoyl, propanoyl, i-propanoyl, n-butanoyl, i-butanoyl or t-butanoyl, preferably acetyl.

The term “hydroxy-protecting group as used for R² denotes groups which intended to protect a hydroxy group and include ester- and ether-forming groups, in particular modified trityl groups, tetrahydropyranyl, acyl groups, carbamoyl, benzyl and silyl ethers (e.g. TBS, TBDPS) groups. Further examples of these groups are found in T. W. Greene and P. G. M. Wuts, “Protective Groups in Organic Synthesis”, 2nd ed., John Wiley & Sons, Inc., New York, N.Y., 1991, chapters 2-3; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981.

Preferred are acid sensitive hydroxyl groups, more preferred alkoxy-modified trityl groups. Modified in this context mean the substitution of one, two or three of the phenyl rings by one or more C₁₋₆-alkoxy groups, preferably methoxy groups.

The most preferred hydroxy protecting group is 4,4′-dimethoxytrityl (DMT).

The term “amino-protecting group” denotes groups intended to protect an amino group and includes C₁₋₆-alkanoyl, benzoyl, benzyloxycarbonyl, carbobenzyloxy (CBZ or Z), 9-fluorenylmethyloxycarbonyl (FMOC), p-methoxybenzyloxycarbonyl, p-nitrobenzyloxycarbonyl, t-butoxycarbonyl (BOC), and trifluoroacetyl, dimethylformamidine (dmf). Further examples of these groups are found in T. W. Greene and P. G. M. Wuts, “Protective Groups in Organic Synthesis”, 2nd ed., John Wiley & Sons, Inc., New York, N.Y., 1991, chapter 7; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981.

Preferred amino protecting groups are selected from C₁₋₆-alkanoyl, benzoyl (bz) or dimethylformamidine (dmf).

The term “nucleobase” or “modified nucleobase” used for R¹ stands for the five nucleobases adenine (A), cytosine (C), guanine (G), thymine (T) and Uracil (U) and for modifications thereof.

The term “salt” in the context of the LNA-dicarboxylic acid derivatives of the present invention of the formula I the present invention stands for a salt from an inorganic or organic base.

Inorganic salts typically are alkali- or earth alkali salts such as sodium-, potassium-, magnesium- or calcium salts, preferably sodium or potassium salts from an inorganic alkali—or earth alkali hydroxide base or from an organic alkali- or earth alkali alcoholate.

Organic salts typically are ammonium salts from an organic amine, typically from an aliphatic amine or an aromatic amine, preferably from a tertiary amine preferably a tri-C₁₋₄-alkylamine or pyridine more preferably triethylamine or pyridine Organic salts are preferred over the inorganic salts.

The LNA-dicarboxylic acid derivatives of formula I can occur in the form of any mixture of the free acid and the salt from the inorganic or organic base.

A typical modified nucleobase is 5-methyl cytosine (5-MeC).

In a preferred embodiment R¹ is an optionally modified and/or an amino group protected adenine (A), cytosine (C), 5-methyl cytosine (5-MeC), guanine (G) or thymine (T), preferably an amino group protected adenine (A), an amino group protected cytosine (C), an amino group protected 5-methyl cytosine (5-MeC), an amino group protected guanine (G) or thymine (T).

While the typical amino protecting groups have been defined above, the preferred amino protecting group for adenine (A), cytosine (C) and 5-methyl cytosine (5-MeC) is benzoyl, for guanine (G) is isobutanoyl (isobutyryl) or dimethylformamidine (dmf).

In a preferred embodiment the LNA-dicarboxylic acid derivatives of the present invention have the formula I

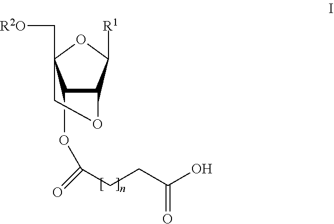

or ammonium salts from an organic amine thereof,

wherein
R¹ is an optionally modified and/or an amino group protected adenine (A), cytosine (C), 5-methyl cytosine (5-MeC), guanine (G) or thymine (T), preferably an amino group protected adenine, an amino group protected cytosine (C), an amino group protected 5-methyl cytosine (5-MeC), an amino group protected guanine (G) or thymine (T).
R² is an acid sensitive hydroxy protecting group selected from an alkoxy-modified trityl group;
n is an integer from 1 to 5.

In a further preferred embodiment the LNA-dicarboxylic acid derivatives of the present invention have the formula I

or ammonium salts from tertiary amines selected from a tri-C₁₋₄-alkylamine, more preferably triethylamine or from pyridine thereof,

wherein;
R¹ is benzoyl protected adenine (A), benzoyl protected cytosine (C), benzoyl protected 5-methyl cytosine (5-MeC), isobutanoyl or dimethylformamidine protected guanine (G) or thymine (T);
R² is 4,4′-dimethoxytrityl (DMT);
n is 1.

The most preferred LNA-dicarboxylic acid derivatives of the present invention have the formula I and are characterized by the following definitions
R¹=benzoyl protected adenine (A); R²=4,4′-dimethoxytrityl (DMT); n=1; in the form of the triethylammonium or pyridinium salt
R¹=benzoyl protected cytosine (C); R²=4,4′-dimethoxytrityl (DMT); n=1; in the form of the triethylammonium or pyridinium salt
R¹=benzoyl protected 5-methyl cytosine (5-MeC); R²=4,4′-dimethoxytrityl (DMT); n=1; in the form of the triethylammonium or pyridinium salt
R¹=isobutanoyl protected guanine (G); R²=4,4′-dimethoxytrityl (DMT); n=1; in the form of the triethylammonium or pyridinium salt
R¹=dimethylformamidine protected guanine (G); R²=4,4′-dimethoxytrityl (DMT); n=1; in the form of the triethylammonium or pyridinium salt
R¹=thymine (T); R²=4,4′-dimethoxytrityl (DMT); n=1 in the form of the triethylammonium or pyridinium salt.

The preferred LNA-dicarboxylic acid derivatives of the present invention as outlined above can either occur as free acids, or in the form of the ammonium salts as described above or also as any mixture of the ammonium salts and the free acid.

The process for the preparation of LNA-dicarboxylic acid derivatives of formula I comprises the reaction of an LNA alcohol of formula II

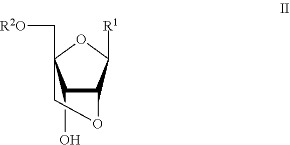

wherein R¹ and R² are as above, with a C₂₋₆-dicarboxylic acid anhydride to form the LNA-dicarboxylic acid derivatives of formula I in the presence of a base and an organic solvent.

LNA alcohols of formula II are as a rule commercially available or can be prepared according to Wengel et al. Tetrahedron 54 (1998) 3607-3630.

Likewise the C₂₋₆-dicarboxylic acid anhydrides are commercial available compounds. Typical C₂₋₆-dicarboxylic acid anhydride is succinic anhydride.

Usually, in an initial step the starting material is freed from residual water under azeotropic conditions with a suitable organic solvent such as with toluene.

Suitable bases have been described above, but preferably organic amines are used.

Preferably a tertiary amine, more preferably a tri-C₁₋₄-alkylamine or pyridine even more preferably triethylamine or pyridine is the base typically applied.

Suitable organic solvent is a halogenated hydrocarbon such as dichloromethane.

The reaction is conveniently performed under inert gas atmosphere at a reaction temperature of 10° C. to 40° C.

Upon completion of the reaction, typically after about 2 h, the ammonium salt can be obtained via an extraction process, suitably applying the solvent used for the reaction.

Removal of the solvent from the combined organic phases and optional further purification by chromatography the respective ammonium salt of the LNA-dicarboxylic acid derivatives of formula I can be delivered in a yield of 94-99.9% and a HPLC purity of 97-99.5 area-% (excluding residual toluene).

The LNA-dicarboxylic acid derivatives of formula I can also be obtained in the form of the free acid. This can be accomplished by converting a salt of the LNA-dicarboxylic acid derivatives of the formula I according to the present invention with hydrochloric acid or an organic acid such as with citric acid in a suitable organic solvent such as in dichloromethane or ethylacetate.

In a further embodiment of the invention the LNA-dicarboxylic acid derivatives of the formula I can be used for the preparation of a preloaded solid support of the formula III

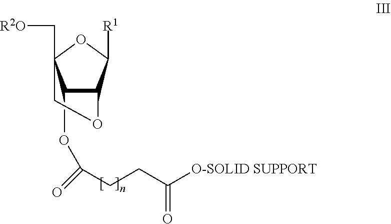

wherein R¹, R² and n are as above and SOLID SUPPORT is a solid support material suitable for oligonucleotide synthesis.
Suitable solid support materials are well described for instance in Guzaev, A. P. Solid-phase supports for oligonucleotide synthesis. In: Current protocols in nucleic acid chemistry. (John Wiley & Sons, Inc.) (2013), Chapter 3, Unit 3.1., pp. 3.1.1-3.1.60. Typical commercial solid supports are the Primer Support 5G series from GE Healthcare or the NittoPhase® HL solid supports from Nitto Denko.
The definitions and preferences provided above for R¹, R² and n likewise apply for the preloaded solid support of the formula III.
In another embodiment the invention relates to preloaded solid support of the formula III

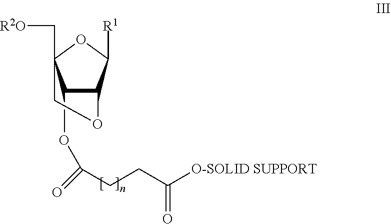

wherein R¹, R², n and SOLID SUPPORT are as defined above. The preferences provided above for R¹, R², n and SOLID SUPPORT likewise apply for the preloaded solid support of the formula III.
In still another embodiment of the invention the preloaded solid support of the formula III

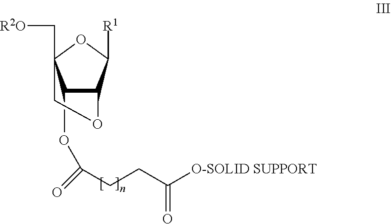

wherein R¹, R², n and SOLID SUPPORT are as above can be used as start material for the oligonucleotide synthesis. The definitions and preferences provided above for R¹, R², n and SOLID SUPPORT likewise apply for the preloaded solid support of the formula III.
The LNA-dicarboxylic acid derivatives of formula I can be linked to the solid support by methods known to the skilled in the art and for instance as described in the Int. Patent Publication WO 1992/006103.

The principles of the oligonucleotide synthesis are well known in the art und well described in literature and public fora like Wikipedia (see e.g. Oligonucleotide synthesis; Wikipedia, the free encyclopedia; https://en.wikipedia.org/wiki/Oligonucleotide_synthesis, of Mar. 15, 2016).

Larger scale oligonucleotide synthesis nowadays is carried automatically using computer controlled synthesizers.

As a rule oligonucleotide synthesis is a solid-phase synthesis, wherein the oligonucleotide being assembled is covalently bound, via its 3′-terminal hydroxy group, to a solid support material and remains attached to it over the entire course of the chain assembly. Suitable solid supports are described above.

Preferably the oligonucleotide consists of optionally modified DNA or LNA nucleoside monomers or combinations thereof and is 10 to 25 nucleotides in length.

In view of the fact that the preloaded solid support of the formula III comprises a LNA nucleoside the 3′ terminal nucleoside of the oligonucleotide chain produced is always an LNA nucleoside.

The oligonucleotide synthesis in principle is a stepwise addition of nucleotide residues to the 5′-terminus of the growing chain until the desired sequence is assembled.

As a rule each addition is referred to as a synthetic cycle and in principle consists of the chemical reactions

a₁₎ de-blocking the protected hydroxyl group on the pre-loaded solid support of formula III,

a₂) coupling the first nucleoside as activated phosphoramidite with the free hydroxyl group on the solid support,

a₃) oxidizing or sulfurizing the respective P-linked nucleoside to form the respective phosphotriester (P═O) or the respective phosphorothioate (P═S);

a₄) optionally, capping any unreacted hydroxyl groups on the solid support;

a₅) de-blocking the 5′ hydroxyl group of the second nucleoside attached to the solid support;

a₆) coupling the third nucleoside as activated phosphoramidite to form the respective P-linked trimer;

a₇) oxidizing or sulfurizing the respective P-linked di-nucleoside to form the respective phosphotriester (P═O) or the respective phosphorothioate (P═S);

a₈) optionally, capping any unreacted 5′ hydroxyl groups;

a₉) repeating the previous steps a₅ to a₈ until the desired sequence is assembled;

a₁₀) global deprotection to obtain the crude oligonucleotide which can be further purified by typical oligonucleotide purification techniques such as with chromatography and/or ultrafiltration and/or lyophilization

The following examples shall illustrate the invention without limiting it.

### EXAMPLES

### Abbreviations

EtOAc=Ethylacetate
MeCN=Acetonitrile
MeOH=Methanol
DCM=Dichloromethane
DMAP=4-Dimethylaminopyridine
TEA=Triethylamine
THF=Tetrahydrofurane
TLC=Thin layer chromatography
rt=Room temperature
eq=Equivalent

### Process Scheme

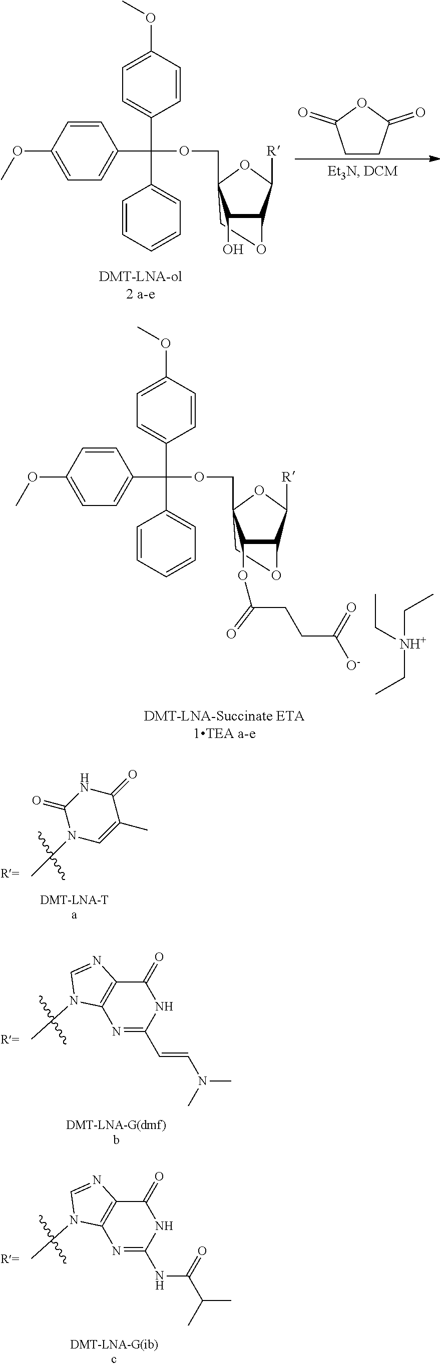

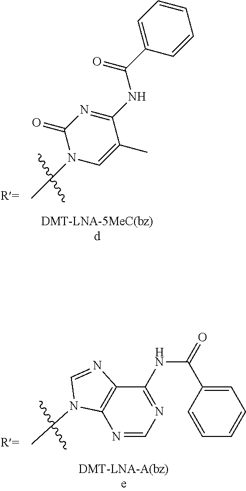 [SMILES: *n1cc(C)c(NC(=O)c2ccccc2)nc1=O ; *n1cnc2c(NC(=O)c3ccccc3)ncnc21]

### General Procedure

A round-bottom flask was charged with nucleoside 2 (1 eq) and succinic anhydride (1.5 eq). For azeotropic removal of residual water, the mixture was suspended in abs. toluene (20 mL per 1 g of 2) at rt. Next volatiles were stripped off in vacuo. This procedure was repeated once, then toluene was stripped off to yield a solid but not fully dried residue. Next operations were all carried out under argon atmosphere. To the above's formed solid, abs. DCM (18 mL per 1 g of 2) was added. Then TEA (3.0 eq rel to 2a, 5.0 eq rel to 2b) was added in one portion and mixture was stirred at rt whereby after 30 min a clear reaction mixture was obtained. The conversion was determined by TLC or LC-MS (see Example 1, Table 1 for 1a.TEA and Example 2, Table 2 for 1b.TEA.

Usually the conversions were complete after 2 h, but in all cases mixtures were additionally stirred for additional 1 h at rt. The reaction mixture was then poured to a triethylammonium phosphate buffer solution (15 mL of buffer per 1 g of 2. Buffer solution preparation: Mixing 85% aq. H₃PO₄ (8.5 mL, 1 eq), TEA (26 mL, 1.5 eq) and water (465.5 mL)). The formed layers were separated and the aqueous layer was additionally extracted with DCM (2×10 mL per 1 g of 2). The combined organic layers were filtered through a pad of anhydrous Na₂SO₄ and concentrated under vacuum. The crude product, obtained as a white foam, was further purified by filtration through a silica column (20 g of silica per 1 g of the crude product; eluent: EtOH in DCM from 2.5% to 10%, the mixture is further supplemented with 3 vol % of TEA). The target fractions were concentrated to dryness, immediately suspended in abs. toluene and co-evaporated in vacuo. Co-evaporation was repeated one more time.

### Example 1: Preparation of 1a.TEA

8.6 g of 2a was converted into 1a.TEA as described in the General Procedure to yield 11.3 g of 1a.TEA in 96% yield and 99.1 HPLC area-% purity (excluding 0.67 mol eq residual toluene) (column: Apollo C-18 (10 μm) (4.6×250 mm) 10.65 min; mobile phase: gradient 40% MeCN+60% of 0.1% aq. H₃PO₄; flow rate 1.0 mL/min; column temperature 40° C.; detection: 210 nm, 254 nm; sample concentration: 1 mg/mL in MeCN; injection volume: 0.002 mL).

LC-MS ESI (m/z): 671.2 [M−H]

¹H-NMR (D4-MeOH, 400 MHz): δ 7.77-7.73 (m, 1H), 7.50-7.43 (m, 2H), 7.38-7.28 (m, 6H), 7.26-7.21 (m, 1H), 6.91-6.85 (m, 4H), 5.62 (s, 1H), 5.21 (s, 1H), 4.56 (s, 1H), 3.89-3.79 (m, 2H), 3.78 (s, 6H), 3.57 (d, J=11.2 Hz, 1H), 3.47 (d, J=11.2 Hz, 1H), 2.94 (q, J=7.3 Hz, 6H), 2.64-2.46 (m, 2H), 2.46-2.36 (m, 2H), 1.61 (d, J=1.2 Hz, 3H), 1.29 (t, J=7.3 Hz, 9H).

TABLE 1

| Method | Sorbent | Eluent | Retention Time | Detection |
|---|---|---|---|---|
| TLC | Merck silica gel | DCM/MeOH 9:1 | R_{f} = 0.55 (2a) | UV 254 nm |
| | TLC plates | incl one drop | R_{f} = 0.25 (1a · TEA) | heating to |
| | UV254 | TEA | | yellow stain |
| LC-MS | Acquity UPLC | MeCN in 0.1% | Rₜ = 3.62 min (1a · TEA) | UV 244 nm |
| | BEH C₁₈ | aq. HCO₂H from | | ESI⁻: 1a · TEA |
| | | 5% to 100% | | [M − H] = 671.2 m/z |

### Example 2: Preparation of 1b.TEA

8.9 g of 2b was converted into 1b.TEA as described in the General Procedure to yield 11.0 g of 1b.TEA in 94% yield and 97.2 HPLC area-% purity (excluding 0.75 mol eq residual toluene) (column: Apollo C-18 (10 μm) (4.6×250 mm) 9.18 min; mobile phase: gradient 40% MeCN+60% of 0.1% aq. H₃PO₄; flow rate 1.0 mL/min; column temperature 40° C.; detection: 210 nm, 254 nm; sample concentration: 1 mg/mL in MeCN; injection volume: 0.002 mL). LC-MS ESI (m/z): 753.3 [M+H]⁺

¹H-NMR (D4-MeOH, 400 MHz): δ 8.70 (s, 1H), 7.98 (s, 1H), 7.44-7.39 (m, 2H), 7.32-7.15 (m, 7H), 6.87-6.81 (m, 4H), 6.00 (s, 1H), 5.85 (s, 1H), 4.80 (s, 1H), 4.01-3.89 (m, 2H), 3.76 (s, 6H), 3.53-3.74 (m, 2H), 3.15 (q, J=7.3 Hz, 6H), 3.09 (s, 3H), 3.07 (s, 3H), 2.63-2.46 (m, 2H), 2.45-2.33 (m, 2H), 1.26 (t, J=7.3 Hz, 9H).

TABLE 2

| Method | Sorbent | Eluent | Retention Time | Detection |
|---|---|---|---|---|
| TLC | Merck silica gel | DCM/MeOH 9:1 | R_{f} = 0.55 (2b) | UV 254 nm |
| | TLC plates | incl one drop | R_{f} = 0.25 (1b · TEA) | heating to yellow stain |
| | UV254 | TEA | | |
| LC-MS | Acquity UPLC | MeCN in 0.1% | Rₜ = 3.40 min (1b · TEA) | UV 303 nm |
| | BEH C₁₈ | aq. HCO₂H from | | ESI⁺: 1b · TEA |
| | | 5% to 100% | | [M + H] = 753.3 m/z |
| | | | | ESI⁻: 1b · TEA |
| | | | | [M − H] = 751.3 m/z |

### Example 3: Preparation of 1c.TEA

10 g of 2c was converted into 1c.TEA as described in the General Procedure to yield 13.6 g of 1c.TEA in 98% yield and 98.6 HPLC area-% purity (excluding 1.20 mol eq residual toluene) (column: Apollo C-18 (10 μm) (4.6×250 mm) 11.00 min; mobile phase: gradient 40% MeCN+60% of 0.1% aq. H₃PO₄; flow rate 1.0 mL/min; column temperature 40° C.; detection: 210 nm, 254 nm; sample concentration: 1 mg/mL in MeCN; injection volume: 0.002 mL).

LC-MS ESI (m/z): 768.5 [M-TEA+H]⁺

¹H-NMR (D4-MeOH, 400 MHz): δ 8.11 (s, 1H), 7.46-7.40 (m, 2H), 7.34-7.26 (m, 4H), 7.25-7.06 (m, 5H), 6.90-6.82 (m, 4H), 5.99 (s, 1H), 5.35 (s, 1H), 4.85 (s, 1H), 3.96 (q, J=8.3 Hz, 2H), 3.76 (s, 6H), 3.56-3.47 (m, 2H), 3.16 (q, J=7.3 Hz, 6H), 2.71 (hept, J=6.8 Hz, 1H), 2.61-2.38 (m, 4H), 1.27 (t, J=7.3 Hz, 9H), 1.21 (d, J=6.9 Hz, 6H).

### Example 4: Preparation of 1d.TEA

10 g of 2d was converted into 1d.TEA as described in the General Procedure to yield 14.0 g of 1d.TEA in >99% yield and 99 HPLC area-% purity (excluding 0.50 mol eq residual toluene) (column: Apollo C-18 (10 μm) (4.6×250 mm) 12.89 min; mobile phase: gradient 40% MeCN+60% of 0.1% aq. H₃PO₄; flow rate 1.0 mL/min; column temperature 40° C.; detection: 210 nm, 254 nm; sample concentration: 1 mg/mL in MeCN; injection volume: 0.002 mL).

LC-MS ESI (m/z): 775.5 [M-TEA+H]⁺

¹H-NMR (D4-MeOH, 400 MHz): δ 8.22 (s, 1H), 8.19 (s, 1H), 7.98 (s, 1H), 7.59-7.52 (m, 1H), 7.51-7.42 (m, 4H), 7.40-7.32 (m, 4H), 7.28-7.09 (m, 5H), 6.95-6.84 (m, 4H), 5.69 (s, 1H), 5.23 (s, 1H), 4.64 (s, 1H), 3.87 (dd, J=15.6 Hz, J=8.2 Hz, 2H), 3.79 (s, 6H), 3.55 (dd, J=34.1 Hz, J=11.3 Hz, 2H), 3.13 (q, J=7.3 Hz, 6H), 2.67-2.37 (m, 4H), 1.83 (s, 3H), 1.27 (t, J=7.3 Hz, 9H).

### Example 5: Preparation of 1e.TEA

10 g of 2c was converted into 1c.TEA as described in the General Procedure to yield 14.2 g of 1c.TEA in >99% yield and 99.2 HPLC area-% purity (excluding 1.00 mol eq residual toluene) (column: Apollo C-18 (10 μm) (4.6×250 mm) 5.17 min; mobile phase: gradient 40% MeCN+60% of 0.1% aq. H₃PO₄; flow rate 1.0 mL/min; column temperature 40° C.; detection: 210 nm, 254 nm; sample concentration: 1 mg/mL in MeCN; injection volume: 0.002 mL).

LC-MS ESI (m/z): 786.5 [M-TEA+H]⁺

¹H-NMR (D4-MeOH, 400 MHz): δ 8.73 (s, 1H), 8.55 (s, 1H), 8.11-8.05 (m, 2H), 7.68-7.61 (m, 1H), 7.59-7.52 (m, 2H), 7.47-7.42 (m, 2H, Ar H), 7.37-7.26 (m, 5H), 7.25-7.07 (m, 3H, Ar H), 6.91-6.82 (m, 4H), 6.24 (s, 1H), 5.44 (s, 1H), 4.93 (s, 1H), 4.02 (dd, 2H, J=16.5 Hz, J=8.3 Hz), 3.77 (s, 6H), 3.57 (br, 2H), 3.12 (q, J=7.3 Hz, 6H), 2.65-2.35 (m, 4H), 1.25 (t, J=7.3 Hz, 9H).

### Example 6: Preparation of 1e.TEA and 1e (Acid Form)

Compound 2e (6.71 g, 9.80 mmol) was dissolved in dry THF (70 mL) and concentrated under vacuum at 35° C. The solid was then further dried under vacuum for 4 h at ambient temperature and dissolved in dry EtOAc (50 mL) and TEA (1.60 mL, 11.7 mmol), DMAP (0.30 g, 2.4 mmol) and succinic anhydride (1.37 g, 13.7 mmol) were sequentially added to the nucleoside solution. The reaction mixture was then heated at 50° C. for 16 hours. The reaction mixture was diluted with EtOAc (100 mL) and hexane (5 mL) and extracted with 10% citric acid solution (3×50 mL). The organic phase was extracted with 5% NaHCO₃ solution (4×50 mL), diluted with EtOAc (150 mL) and further extracted with 10% citric acid solution (2×75 mL) and water (2×75 mL). The combined citric acid and water fractions were back extracted with EtOAc (2×50 mL). The combined organic phases were dried (Na₂SO₄) and concentrated under reduced pressure to obtain the succinate 1e (acid form) as a white foam.

¹H NMR (600 MHz, DMSO-d6) δ ppm 12.27 (br s, 1H), 11.28 (s, 1H), 8.79 (s, 1H), 8.51 (s, 1H), 8.06 (br d, J=7.5 Hz, 2H), 7.64-7.70 (m, 1H), 7.53-7.61 (m, 2H), 7.39 (br d, J=7.6 Hz, 2H), 7.32 (br t, J=7.7 Hz, 2H), 7.23-7.28 (m, 4H), 7.24 (br s, 1H), 6.90 (br d, J=8.6 Hz, 4H), 6.23 (s, 1H), 5.58 (s, 1H), 4.93 (s, 1H), 4.02-4.07 (m, 2H), 3.89 (br d, J=8.5 Hz, 1H), 3.74 (s, 6H), 3.53 (br d, J=11.2 Hz, 1H), 3.41 (br d, J=11.2 Hz, 1H), 2.40-2.45 (m, 2H).

The solid was redissolved in MeCN (70 mL) and TEA (6.8 mL, 49 mmol) was added. After stirring 10 minutes, the mixture was concentrated under vacuum at 35° C. for 10 minutes and the remainder was dried under vacuum at ambient temperature overnight to obtain 1e.TEA salt (7.45 g of succinate×0.6 TEA, 90% yield, HPLC purity >98%).

Analytical data for 1e.TEA were in agreement with those reported in Example 5

1. A LNA-dicarboxylic acid derivative comprising formula I: 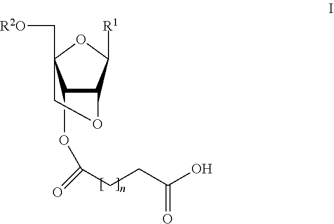
or a salt thereof,
wherein
R¹ is a nucleobase or a modified nucleobase,
R² is a hydroxy protecting group, and
n is an integer from 1 to 5.

2. The LNA-dicarboxylic acid derivative or salt thereof of claim 1, wherein R¹ is an optionally modified and/or an amino group protected adenine (A), cytosine (C), guanine (G) or thymine (T).

3. The LNA-dicarboxylic acid derivative or salt thereof of 2, wherein R¹ is an amino group protected adenine (A), an amino group protected cytosine (C), an amino group protected 5-methyl cytosine (5-MeC), an amino group protected guanine (G) or is thymine.

4. The LNA-dicarboxylic acid derivative or salt thereof of claim 3, wherein the amino protecting group is selected from C₁₋₆-alkanoyl, benzoyl (bz) or dimethylformamidine (dmf).

5. The LNA-dicarboxylic acid derivative or salt thereof of claim 1, wherein R² is an acid sensitive hydroxyl protecting group selected from an alkoxy-modified trityl group.

6. The LNA-dicarboxylic acid derivative or salt thereof of claim 1, wherein n is 1.

7. A salt of the LNA-dicarboxylic acid derivative of claim 1, wherein the salt of the LNA-dicarboxylic acid derivative is a salt from an inorganic or organic base.

8. A salt of the LNA-dicarboxylic acid derivative of claim 1, wherein the salt of the LNA-dicarboxylic acid derivative is an ammonium salt of an organic amine.

9. A salt of the LNA-dicarboxylic acid derivative of claim 8, wherein the salt of the LNA-dicarboxylic acid derivative is an ammonium salt of a tertiary amine.

10. The LNA-dicarboxylic acid derivative or salt thereof of claim 1, comprising a mixture of free acid and salt from the inorganic or organic base.

11. A process for the preparation of a LNA-dicarboxylic acid derivative or salt thereof of formula I, the process comprising the reaction of an LNA alcohol of formula II: 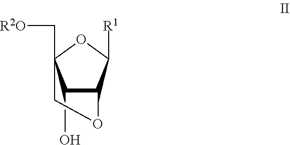
wherein
R¹ is a nucleobase or a modified nucleobase; and
R² is a hydroxy protecting group:
with a C₂₋₆-dicarboxylic acid anhydride in the presence of a base and an organic solvent to form the LNA-dicarboxylic acid derivative or salt thereof of formula I.

12. The process of claim 11, wherein the C₂₋₆-dicarboxylic acid anhydride is succinic anhydride.

13. The process of claim 11, wherein the base is an organic amine.

14. The process of claim 11, wherein the organic solvent is a halogenated hydrocarbon.

15. A method of synthesizing a a preloaded solid support of the formula III: 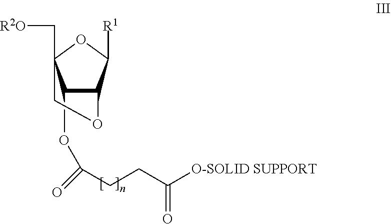
the method comprising contacting a LNA-dicarboxylic acid derivative or salt thereof of formula I of claim 1 and a SOLID SUPPORT comprising a solid support material suitable for oligonucleotide synthesis.

16. A preloaded solid support of the formula III: 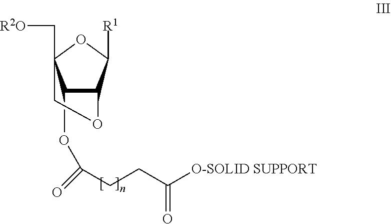
wherein
R¹ is a nucleobase or a modified nucleobase;
R² is a hydroxy protecting group; and
n is an integer from 1 to 5; and
SOLID SUPPORT comprises a solid support material suitable for oligonucleotide synthesis.

17. A method for the synthesis of a 3′-terminal LNA nucleoside, the method comprising stepwise addition of nucleotide residues to the 5′ terminus of a preloaded solid support of the formula III of claim 16.

18. The LNA-dicarboxylic acid derivative or salt thereof of claim 5, wherein R² is 4,4′-dimethoxytrityl.

19. The process of claim 13, wherein the base is a tertiary amine, a tri-C₁₋₄-alkylamine, or pyridine.

20. The process of claim 19, wherein the base is triethylamine or pyridine.
